(19) United States
(12) Patent Application Publication
(10) Pub. No.: US 2018/0280392 A1
(43) Pub. Date: Oct. 4, 2018

(54) PHARMACEUTICAL PREPARATION COMPRISING CYCLIN INHIBITOR AND PREPARATION METHOD THEREOF

(71) Applicant: Jiangsu Hansoh Pharmaceutical Group Co., Ltd., Lianyungang, Jiangsu (CN)

(72) Inventors: Jin ZENG, Lianyungang, Jiangsu (CN); Ruijun WANG, Lianyungang, Jiangsu (CN); Xiaolei WANG, Lianyungang, Jiangsu (CN)

(21) Appl. No.: 15/524,394

(22) PCT Filed: Nov. 6, 2015
(86) PCT No.: PCT/CN2015/093953

§ 371 Date: May 4, 2017

(30) Foreign Application Priority Data
Nov. 7, 2014 (CN) 201410623810.2

(51) Int. Cl. A61K 31/519; A61K 9/20; A61K 9/28; A61K 9/48

(52) U.S. Cl. CPC A61K 31/519; A61K 9/2009; A61K 9/2018; A61K 9/2054; A61K 9/4866; A61K 9/28; A61K 9/485; A61K 9/4858; A61K 9/2059

(57) ABSTRACT

Disclosed is a pharmaceutical preparation having 6-acetyl-8-cyclopentyl-5-methyl-2-(5-piperazine-1-yl-pyridine-2-yl amino group)-8H-pyrido[2,3-d]pyrimidine-7-one or salt thereof as an active ingredient, the salt comprising hydrochloride or isethionate, and the dosage form thereof comprising tablets and capsules both having good stability and excellent dissolution performance.

### FIELD OF THE INVENTION

The present invention relates to the field of pharmaceutical preparation, and specifically relates to a pharmaceutical preparation for use as a cyclin inhibitor and a preparation method thereof.

### BACKGROUND OF THE INVENTION

Cyclin-dependent kinase (CDK) has 13 members in total, which all belong to the serine/threonine protein kinase family, and has key functions such as promoting the phase transition of cell cycle, initiating DNA synthesis and regulating cell transcription and the like, depending on the combination with a cyclin.

CDKs play a key role in the proliferation and death of all cells, including healthy cells and tumor cells. Broad-spectrum CDK inhibitors can hardly exhibit high therapeutic window on patients, especially on patients who have not undergone a gene screening. The toxicity will be severe when the dosage is too high, while the efficacy will be negligible when the dosage is too low. Therefore, it is very important to selectively inhibit some CDKs. Of course, since most of the CDK subtypes have relatively similar chemical structures, how to improve the selectivity of CDK inhibitors is another challenge.

The advantages of using CDK4/6 as an anti-tumor target are as follows: (1) the inhibitors of CDK4/6 do not exhibit cytotoxicities of “pan-CDK inhibitors,” such as myelosuppression and intestinal responses; and (2) the increase of cell cyclin D level or the inactivation of P161NK4a can improve the sensitivity of cells to drugs. The aforementioned phenomena are presented in tumor cells relative to normal cells, therefore the targeted property of drugs will be increased to a certain extent. The compound of formula I is a targeted CDK4/6 inhibitor, which can selectively inhibit cyclin-dependent kinase 4 and 6 (CDK4/6), restore cell cycle control, and block the proliferation of tumor cells. It acts on MDA-MB-435 breast cancer cells, and can effectively reduce the phosphorylation of retinoblastoma tumor suppressor (RB) on Ser780 and Ser795 sites, and the IC₅₀ are 66 nM and 63 nM, respectively.

Breast cancer is one of the most common malignant tumors of women, with high incidence rates and invasiveness, but the course of progress is slow. The data published by the International Cancer Research Center showed that about 1.67 million cases were newly diagnosed around the world in 2012, which accounted for 25% of all cancers. According to the Survey of Epidemiology and End Results (SEER) of the U.S. National Cancer Institute, the estimated incidence rate of breast cancer was 123.8 per one hundred thousand people in the U.S. in 2013, and the incidence rate of breast cancer of Asian females was 94.5 per one hundred thousand people in 2005-2009. There is no official data in China, but it is estimated that the estrogen or progesterone receptor is positive among 60%-70% of breast cancer patients in China. The incidence rate of estrogen receptor positive breast cancer was thus calculated to be 56.7-66.15 per one hundred thousand people in China in 2005-2009. According to Thomson's prediction, the sales of this product will increase significantly after it comes onto the market, and the sales are expected to reach 2.027 billion U.S. dollars in 2019.

### SUMMARY OF THE INVENTION

The objective of the present invention is to provide a pharmaceutical preparation comprising 6-acetyl-8-cyclopentyl-5-methyl-2-(5-piperazin-1-yl-pyridin-2-yl-amino)-8H-pyrido[2,3-d]pyrimidin-7-one and a salt thereof, and a preparation method thereof. The technical solution of the present invention is achieved as follows:

A pharmaceutical composition, comprising the compound of formula I or the salt thereof, and a pharmaceutically acceptable excipient as a vehicle, wherein the salt comprises hydrochloride or isethionate.

Preferably, the compound of formula I is present in an amount of 10%-80%, preferably 15%-60%, further preferably 20%-40% by weight, relative to the weight of the composition.

Preferably, the composition comprises the compound of formula I or the salt thereof and the excipient, wherein the excipient is one or more selected from the group consisting of a disintegrant, diluent, binder, surfactant, and lubricant, preferably, the weight percentage of each component is as follows:

| | |
|---|---|
| compound or salt thereof | 10-80% |
| disintegrant | 1-25% |
| diluent | 10-80% |
| lubricant | 0.1-5.0% |
| surfactant | 0-5.0% |
| binder | 0-20%. |

Optionally, the composition can also comprise a flavoring agent, a colorant or a coating material, most preferably, the sum of the weight percentages of the aforementioned components is 100%.

Further preferably, in the pharmaceutical composition, the weight percentage of each component is as follows:

| | |
|---|---|
| compound or salt thereof | 15-60% |
| disintegrant | 3-15% |
| diluent | 30-80% |
| lubricant | 0.1-3.5% |
| binder | 0-10% |
| surfactant | 0-5.0%. |

Optionally, the composition can also comprise a flavoring agent, a colorant or a coating material, most preferably, the sum of the weight percentages of the aforementioned components is 100%.

More preferably, in the pharmaceutical composition, the weight percentage of each component is as follows:

| | |
|---|---|
| compound I or salt thereof | 20-40% |
| disintegrant | 3-15% |
| diluent | 55-70% |
| lubricant | 0.1-3.5% |
| binder | 0-8% |
| surfactant | 0-5.0%. |

Preferably, the diluent is at least one selected from the group consisting of starch, powdered sugar, dextrin, lactose, pregelatinized starch, calcium hydrogen phosphate, calcium sulfate, calcium carbonate, mannitol, sorbitol and microcrystalline cellulose. More preferably, the diluent is at least one selected from the group consisting of lactose, microcrystalline cellulose, starch and mannitol, most preferably, the diluent is selected from the group consisting of lactose and microcrystalline cellulose.

Preferably, the weight ratio of lactose to microcrystalline cellulose is 1:2-2:1, preferably 1:1.

Preferably, the binder is at least one selected from the group consisting of starch slurry, hydroxypropyl methylcellulose, hydroxypropyl cellulose, povidone, methyl cellulose, sodium carboxymethyl cellulose, and polyethylene glycol. More preferably, the diluent is at least one selected from the group consisting of hydroxypropyl cellulose and povidone.

Preferably, the disintegrant is at least one selected from the group consisting of croscarmellose sodium, sodium carboxymethyl starch, crospovidone, dry starch, and low-substituted hydroxypropyl cellulose. More preferably, the disintegrant is at least one selected from the group consisting of crospovidone, sodium carboxymethyl starch, and croscarmellose sodium.

Preferably, the lubricant is at least one selected from the group consisting of magnesium stearate, stearic acid, sodium stearyl fumarate, glyceryl behenate, colloidal silica, talc, and silica. More preferably, the lubricant is at least one selected from the group consisting of stearic acid, glyceryl behenate, and colloidal silica.

Preferably, the surfactant is sodium dodecyl sulfate, Tween 80 and poloxamer. More preferably, the surfactant is sodium dodecyl sulfate.

In another aspect, the present invention provides a preparation method of the compound of formula I or the salt thereof. The preparation process comprises wet granulation, dry granulation, direct mixing and the like, and the dosage form comprises a tablet or capsule.

Preferably, the composition is obtained by wet granulation, and the method comprises the following steps of:

(1) pre-mixing the compound of formula I or the salt thereof with a portion of a lubricant in a wet mixing granulator to obtain a pre-mixture;

(2) adding a granulation liquid to granulate the pre-mixture obtained in step (1), preferably, the granulation liquid being water;

(3) drying the granule obtained in step (2) in a fluidized bed dryer or a drying oven;

(4) optionally, dry screening the dry granule obtained in step (3);

(5) mixing the dry granule obtained in step (4) with the rest of excipient(s) to obtain a final mixture;

(6) optionally, filling the mixture obtained in the aforementioned step (5) by a suitable capsule filling machine to prepare a capsule;

(7) optionally, pressing the mixture obtained in the aforementioned step (5) by a suitable tabletting machine to obtain a tablet core; and

(8) optionally, film coating the tablet core obtained in step (7) with a film coating.

Preferably, the composition is obtained by dry granulation, and the method comprises the following steps of:

(1) mixing the compound of formula I or the salt thereof with the majority of excipients including a binder in a hopper mixer to obtain a pre-mixture;

(2) pressing the mixture obtained in step (1) in a suitable roller press machine;

(3) crushing the ribbon obtained during step (2) into a granule by a suitable grinding or screening step;

(4) optionally, mixing the granule obtained in step (3) with the rest of the excipient in a mixer to obtain a final mixture;

(5) optionally, filling the mixture obtained in the aforementioned step (4) by a suitable capsule filling machine to prepare a capsule;

(6) optionally, pressing the mixture obtained in the aforementioned step (4) by a suitable tabletting machine to obtain a tablet core; and

(7) optionally, film coating the tablet core obtained in step (6) with film a coating. The present invention provides a pharmaceutical preparation of 6-acetyl-8-cyclopentyl-5-methyl-2-(5-piperazin-1-yl-pyridin-2-yl-amino)-8H-pyrido[2,3-d]pyrimidin-7-one and a salt thereof, which is stable and suitable for medical applications. The pharmaceutical preparation has excellent dissolution behavior and good stability, which meets the requirements for clinical use, and enables the active ingredient to achieve a good in vivo bioavailability.

### DETAILED DESCRIPTION OF THE INVENTION

The embodiments of the present invention will be described in detail with reference to specific examples. The following examples are only intended to illustrate the present invention, and should not be considered as limiting the scope of the present invention.

### Example 1

1.1 Composition of Unit Formula

| | Formula 1 | Formula 2 | Formula 3 | Formula 4 | Formula 5 | Formula 6 | Formula 7 | Formula 8 |
|---|---|---|---|---|---|---|---|---|
| Compound of | 75 | 75 | 75 | 75 | 75 | 75 | 100 | 125 |
| formula I | | | | | | | | |
| Microcrystalline | 35.2 | / | 28.2 | 54.3 | 18.8 | 37.6 | 93.9 | 117.3 |
| cellulose | | | | | | | | |
| Lactose | 35.2 | 56.4 | 56.4 | 28.2 | 10.8 | 18.8 | 46.9 | 58.7 |
| Starch | 35.2 | 28.2 | / | / | / | / | / | / |
| Hydroxypropyl | 10.5 | 11.5 | 10.5 | / | / | 7.0 | 14 | 17.5 |
| cellulose | | | | | | | | |
| Povidone | / | 20.0 | / | 10.5 | 3.5 | / | / | / |
| Sodium | 10.5 | 5.5 | 31.5 | / | / | 21 | 14 | 17.5 |
| carboxymethyl | | | | | | | | |
| starch | | | | | | | | |
| Crospovidone | / | 5.0 | / | 31.5 | / | / | / | / |
| Croscarmellose | / | / | / | / | 10.5 | / | / | / |
| sodium | | | | | | | | |
| Sodium dodecyl | 4.2 | 4.2 | 4.2 | 6.3 | 0 | 2.8 | 5.6 | 7.0 |
| sulfate | | | | | | | | |
| Talc | 2.1 | 2.1 | 2.1 | 2.1 | 0.7 | 1.4 | 2.8 | 3.5 |
| Magnesium | 2.1 | 2.1 | 2.1 | 2.1 | 0.7 | 1.4 | 2.8 | 3.5 |
| stearate | | | | | | | | |
| Weight | 210 | 210 | 210 | 210 | 120 | 165 | 280 | 350 |

Note:
“/” represents that the corresponding component was not added.

1.2 Preparation

The compound of formula I and the excipients in the aforementioned formulation, except magnesium stearate, in an amount for 1000 tablets were mixed in a hopper mixer, and a wetting agent was added to carry out wet granulation. The granule was dried in a fluidized bed at 45° C. for 10 minutes, and sieved through a 1.0 mm sieve. Magnesium stearate was added, and the mixture was blended for 10 minutes. The content was monitored on line. The mixture was filled in capsules, or pressed into tablets.

1.3 Dissolution Data

The dissolution rate of each formula in 0.1 mol/L HCl is shown in the table below.

| | Formula 1 | Formula 2 | Formula 3 | Formula 4 | Formula 5 | Formula 6 | Formula 7 | Formula 8 |
|---|---|---|---|---|---|---|---|---|
| 10 min | 35 | 40 | 32 | 27 | 22 | 42 | 41 | 37 |
| 15 min | 62 | 66 | 68 | 56 | 55 | 66 | 69 | 65 |
| 30 min | 78 | 82 | 77 | 70 | 73 | 79 | 82 | 79 |
| 45 min | 89 | 91 | 93 | 83 | 80 | 87 | 89 | 83 |
| 60 min | 95 | 96 | 98 | 88 | 91 | 99 | 96 | 95 |

### Example 2

2.1 Composition of Unit Formula

| | Formula | Formula | Formula | Formula |
|---|---|---|---|---|
| | 9 | 10 | 11 | 12 |
| Compound of formula I | 75 | 75 | 100 | 125 |
| Microcrystalline cellulose | 38.7 | 10.8 | 93.9 | 117.3 |
| Lactose | 54.3 | 18.8 | 49.7 | 65.7 |
| Hydroxypropyl cellulose | / | / | / | 17.5 |
| Povidone | 10.5 | 3.5 | 14 | / |
| Sodium carboxymethyl starch | / | 10.5 | 14 | 17.5 |
| Croscarmellose sodium | 21 | / | / | / |
| Sodium dodecyl sulfate | 2.1 | / | 2.8 | / |
| Talc | 2.1 | 0.7 | 2.8 | 3.5 |
| Magnesium stearate | 2.1 | 0.7 | 2.8 | 3.5 |
| Weight | 210 | 120 | 280 | 350 |

Note:
“/” represents that the corresponding component was not added.

2.2 Preparation

The compound of formula I and the excipients in the aforementioned formulation, except magnesium stearate, in an amount for 1000 tablets were mixed in a hopper mixer. The mixture was pressed into a ribbon by using a roller press machine, then the ribbon was crushed into a granule. Magnesium stearate was added, and the mixture was blended for 10 minutes. The content was monitored on line. The mixture was filled in capsules, or pressed into tablets.

2.3 Dissolution Data

| | Formula 9 | Formula 10 | Formula 11 | Formula 12 |
|---|---|---|---|---|
| 10 min | 44 | 38 | 35 | 38 |
| 15 min | 69 | 63 | 64 | 70 |
| 30 min | 76 | 74 | 75 | 81 |
| 45 min | 93 | 90 | 89 | 90 |
| 60 min | 98 | 98 | 93 | 92 |

### Example 3

3.1 Composition of Unit Formula

| | Formula 13 | Formula 14 | Formula 15 |
|---|---|---|---|
| Compound of formula I | 75 | 75 | 75 |
| Microcrystalline cellulose | 71.8 | / | 50 |
| Lactose | 35.9 | 25.8 | 15.7 |
| Hydroxypropyl cellulose | 10.5 | 6.0 | 9.0 |
| Sodium carboxymethyl starch | 10.5 | 6.0 | 5.0 |
| Crospovidone | / | 3.0 | 4.0 |
| Sodium dodecyl sulfate | 2.1 | / | 2.1 |
| Talc | 2.1 | 2.1 | 2.1 |
| Magnesium stearate | 2.1 | 2.1 | 2.1 |
| Weight | 210 | 120 | 165 |

Note:
“/” represents that the corresponding component was not added.

3.2 Preparation

The compound of formula I and the excipients in the aforementioned formulation in an amount for 1000 tablets were mixed in a hopper mixer. The content was monitored on line. The mixture was filled in capsules, or pressed into tablets.

3.3 Dissolution Data

| | Formula 13 | Formula 14 | Formula 15 |
|---|---|---|---|
| 10 min | 40 | 29 | 35 |
| 15 min | 71 | 56 | 64 |
| 30 min | 80 | 70 | 77 |
| 45 min | 93 | 78 | 85 |
| 60 min | 98 | 82 | 90 |

### Example 4

4.1 Composition of Unit Formula

| | Formula 16 | Formula 17 | Formula 18 |
|---|---|---|---|
| Active pharmaceutical | 125 | 125 | 125 |
| ingredient (API)² | | | |
| Microcrystalline cellulose | 124.3 | 64.5 | 27.1 |
| Lactose | 62.2 | 64.6 | 27.1 |
| Hydroxypropyl cellulose | 17.5 | 8.4 | 16.8 |
| Crospovidone | / | 14 | 10.5 |
| Croscarmellose sodium | 17.5 | / | / |
| Magnesium stearate | 3.5 | 3.5 | 3.5 |
| Weight | 350 | 280 | 210 |

Note 1:
“/” represents that the corresponding component was not added.
Note 2:
API represents a hydrochloride or isethionate salt of the compound of formula I.

4.2 Preparation

The API and excipients in the aforementioned formulation, except magnesium stearate, in an amount for 1000 tablets were mixed in a hopper mixer, and a wetting agent was added to carry out wet granulation. The granule was dried in a fluidized bed at 45° C. for 10 minutes, and sieved through a 1.0 mm sieve. Magnesium stearate was added, and the mixture was blended for 10 minutes. The content was monitored on line. The mixture was filled in capsules, or pressed into tablets.

4.3 Dissolution Data

| | Formula 16 | Formula 17 | Formula 18 |
|---|---|---|---|
| 5 min | 70 | 73 | 71 |
| 15 min | 95 | 92 | 91 |
| 30 min | 98 | 97 | 98 |

### Example 5

5.1 Composition of Unit Formula

| | Formula | Formula | Formula | Formula |
|---|---|---|---|---|
| | 19 | 20 | 21 | 22 |
| API² | 75 | 75 | 75 | 100 |
| Microcrystalline cellulose | 74.6 | 37.3 | 34.9 | 74.6 |
| Lactose | 37.3 | 74.6 | 34.9 | 74.6 |
| Povidone | 10.5 | 10.5 | 8.25 | 14 |
| Sodium carboxymethyl starch | / | 10.5 | 5 | 14 |
| Croscarmellose sodium | 10.5 | / | 3.25 | / |
| Magnesium stearate | 2.1 | 2.1 | 1.65 | 2.8 |
| Weight | 210 | 210 | 165 | 280 |

Note 1:
“/” represents that the corresponding component was not added.
Note 2:
API represents a hydrochloride of the compound of formula I (Note: the aforementioned formula is also applicable to isethionate, and the inventor detected that the dissolution effect of isethionate is very similar to that of the hydrochloride used as the API).

5.2 Preparation

The API and excipients in the aforementioned formulation, except magnesium stearate, in amount for 1000 tablets were mixed in a hopper mixer. The mixture was pressed into a ribbon by using a roller press machine, and the ribbon was then crushed into granules. Magnesium stearate was added, and the mixture was blended for 10 minutes. The content was monitored on line. The mixture was filled in capsules, or pressed into tablets.

5.3 Dissolution Data

| | Formula 19 | Formula 20 | Formula 21 | Formula 22 |
|---|---|---|---|---|
| 5 min | 76 | 70 | 68 | 69 |
| 15 min | 99 | 94 | 97 | 91 |
| 30 min | 98 | 99 | 99 | 97 |

### Example 6

6.1 Composition of Unit Formula

| | Formula 23 | Formula 24 | Formula 25 |
|---|---|---|---|
| API² | 75 | 100 | 125 |
| Microcrystalline cellulose | 37.3 | 49.7 | 62.2 |
| Lactose | 74.6 | 99.5 | 124.3 |
| Hydroxypropyl cellulose | 10.5 | 14 | 17.5 |
| Croscarmellose sodium | 10.5 | 14 | 17.5 |
| Magnesium stearate | 2.1 | 2.8 | 3.5 |
| Weight | 210 | 280 | 350 |

Note 1:
“/” represents that the corresponding component was not added.
Note 2:
API represents a hydrochloride salt of the compound of formula I (Note: the aforementioned formula is also applicable to isethionate, and the inventor detected that the dissolution effect of isethionate is very similar to that of the hydrochloride salt used as the API).

6.2 Preparation

The API and excipients in the aforementioned formulation in an amount for 1000 tablets were mixed in a hopper mixer. The content was monitored on line. The mixture was filled in capsules, or pressed into raw tablets, which were then coated. The weight increase of coating was controlled by 3%.

6.3 Dissolution Data

| | Formula 23 | Formula 24 | Formula 25 |
|---|---|---|---|
| 5 min | 62 | 60 | 59 |
| 15 min | 90 | 90 | 94 |
| 30 min | 97 | 95 | 99 |

### Example 7

7.1 Unit Dosage Formula

| | Formula | Formula | Formula | Formula | Formula |
|---|---|---|---|---|---|
| | 26 | 27 | 28 | 29 | 30 |
| Salt of the compound of | 81 | 81 | 81 | 96 | 160 |
| formula I¹ | | | | | |
| Microcrystalline | 47.7 | 72.9 | 106.5 | 99 | 67 |
| cellulose | | | | | |
| Lactose | 47.7 | 72.9 | 106.5 | 99 | 67 |
| Hydroxypropyl cellulose | 10.5 | 13.5 | 17.5 | 17.5 | 17.5 |
| Croscarmellose sodium | 10.5 | 13.5 | 17.5 | 17.5 | 17.5 |
| Sodium dodecyl sulfate | 6.3 | 8.1 | 10.5 | 10.5 | 10.5 |
| Colloidal silica | 4.2 | 5.4 | 7 | 7 | 7 |
| Magnesium stearate | 2.1 | 2.7 | 3.5 | 3.5 | 3.5 |
| Weight | 210 | 270 | 350 | 350 | 350 |

Note 1:
The conversion coefficient of the compound of formula I to the hydrochloride salt is 447.53/484.03 = 92.4%, i.e., 75 mg of the compound of formula I corresponds to 81 mg of the hydrochloride salt of the compound of formula I; the conversion coefficient of the compound of formula I to isethionate is 447.53/573.53 = 78.0%, i.e., 75 mg of compound of formula I corresponds to 96 mg of the isethionate salt of the compound of formula I; other specifications are converted correspondingly.

7.2 Preparation

For formulas 26-28, the API and excipients in the formulation in an amount for 1000 tablets were mixed in a hopper mixer. The content was monitored on line. The mixture was filled in capsules. For formulas 29-30, after mixing, the mixture was directly pressed into raw tablets, which were then coated. The weight increase of coating was controlled by 3%.

7.3 Dissolution Data

| | Formula | Formula | Formula | Formula | Formula |
|---|---|---|---|---|---|
| | 26 | 27 | 28 | 29 | 30 |
| 5 min | 40 | 60 | 82 | 85 | 40 |
| 15 min | 82 | 85 | 95 | 95 | 73 |
| 30 min | 97 | 100 | 103 | 98 | 99 |

### Example 8

8.1 Unit Dosage Formula

| | Formula | Formula | Formula | Formula | Formula |
|---|---|---|---|---|---|
| | 31 | 32 | 33 | 34 | 35 |
| Compound of formula I | 75 | 100 | 100 | 125 | 125 |
| Microcrystalline | 86.7 | 77.1 | 154.1 | 144.5 | 56.3 |
| cellulose | | | | | |
| Lactose | 86.7 | 154.1 | 77.1 | 144.5 | 232.7 |
| Sodium carboxymethyl | 13.5 | 18 | 18 | 22.5 | 22.5 |
| starch | | | | | |
| Colloidal silica | 5.4 | 7.2 | 7.2 | 9.0 | 9.0 |
| Magnesium stearate | 2.7 | 3.6 | 3.6 | 4.5 | 4.5 |
| Weight | 270 | 360 | 360 | 450 | 450 |

8.2 Preparation

For formulas 31-32, the API and excipients in the formulation in an amount for 1000 tablets were mixed in a hopper mixer. The content was monitored on line. The mixture was filled in capsules. For formulas 33-34, after mixing, the mixture was directly pressed into raw tablets, which were then coated. The weight increase of coating was controlled by 3%. For formula 35, the compound of formula I and other excipients, except magnesium stearate, were mixed in a hopper mixer. The mixture was pressed into a ribbon by using a roller press machine, and the ribbon was then crushed into granules. Magnesium stearate was added, and the mixture was blended for 10 minutes. The content was monitored. The mixture was pressed into tablets, which were then coated. The weight increase of coating was controlled by 3%.

8.3 Dissolution data

| | Formula | Formula | Formula | Formula | Formula |
|---|---|---|---|---|---|
| | 31 | 32 | 33 | 34 | 35 |
| 5 min | 85 | 77 | 76 | 83 | 70 |
| 15 min | 94 | 85 | 91 | 95 | 85 |
| 30 min | 98 | 100 | 97 | 99 | 99 |

8.4 Stability Data

The capsules prepared in Example 8 were packaged in a commercially available package, and then placed under a relative humidity of 75%±5% at 40° C.±2° C. for 6 months. The results are shown in Table 1.

### Table 1: Results of Stability Test

| Test | | Formula 31 | | Formula 32 | | Formula 33 | | Formula 34 | | Formula 35 | |
|---|---|---|---|---|---|---|---|---|---|---|---|
| items | | 0 day | 6 M | 0 day | 6 M | 0 day | 6 M | 0 day | 6 M | 0 day | 6 M |
| Content (%) | | 99.52 | 99.78 | 99.46 | 99.41 | 100.23 | 99.43 | 99.51 | 99.42 | 99.51 | 99.42 |
| Total impurity (%) | | 0.18 | 0.17 | 0.19 | 0.18 | 0.18 | 0.19 | 0.17 | 0.18 | 0.17 | 0.18 |
| Dissolution | 5 min | 85 | 87 | 80 | 80 | 80 | 79 | 87 | 82 | 70 | 72 |
| rate | 15 min | 95 | 95 | 90 | 88 | 91 | 89 | 95 | 95 | 85 | 86 |
| (%) | 30 min | 99 | 98 | 100 | 99 | 97 | 98 | 99 | 98 | 99 | 98 |

### Example 9

### 9.1 Unit Dosage Formula

| | Formula | Formula | Formula | Formula | Formula | Formula |
|---|---|---|---|---|---|---|
| | 36 | 37 | 38 | 39 | 40 | 41 |
| Salt of compound of formula I¹ | 81 | 81 | 81 | 160 | 160 | 160 |
| Microcrystalline cellulose | 101 | 67.5 | 135 | 100 | 56.5 | 33 |
| Lactose | 101.5 | 135 | 67.5 | 37.5 | 56.5 | 66 |
| Starch | / | / | / | / | / | / |
| Hydroxypropyl cellulose | / | / | / | / | 17.5 | 17.5 |
| Povidone | 17.5 | 17.5 | 17.5 | / | / | / |
| Sodium carboxymethyl starch | / | / | 35 | 17.5 | 17.5 | / |
| Crospovidone | 35 | 35 | / | / | 17.5 | 17.5 |
| Croscarmellose sodium | / | / | / | 17.5 | / | 35 |
| Sodium dodecyl sulfate | 3.5 | 3.5 | 3.5 | 7.0 | 14.0 | 10.5 |
| Colloidal silica | 3.5 | 3.5 | / | 7.0 | 7.0 | 7.0 |
| Talc | / | / | 3.5 | / | / | / |
| Glyceryl behenate | 7.0 | 7.0 | 7.0 | 3.5 | 3.5 | 3.5 |
| Weight | 350 | 350 | 350 | 350 | 350 | 350 |

Note
¹the conversion coefficient of the compound of formula I to a hydrochloride salt is 447.53/484.03 = 92.4%, i.e., 75 mg of the compound of formula I corresponds to 81 mg of the hydrochloride salt of the compound of formula I; the conversion coefficient of the compound of formula I to an isethionate salt is 447.53/573.53 = 78.0%, i.e., 75 mg of the compound of formula I corresponds to 96 mg of the isethionate salt of the compound of formula I; other specifications are converted correspondingly.

### 9.2 Preparation

Wet granulation was carried out with the salt of the compound of formula I and excipients except lubricant, the granule was sieved after drying, a lubricant was added, and the mixture was blended for 10 minutes. The content was monitored on line. For formulas 36-38, the mixture was filled in capsules; for formulas 39-41, the mixture was pressed into tablets, which were then coated, and the weight increase of coating was controlled by 3%.

### 9.3 Dissolution Data

| | Formula | Formula | Formula | Formula | Formula | Formula |
|---|---|---|---|---|---|---|
| | 36 | 37 | 38 | 39 | 40 | 41 |
| 5 min | 85 | 79 | 77 | 65 | 56 | 50 |
| 15 min | 95 | 89 | 90 | 79 | 80 | 76 |
| 30 min | 102 | 96 | 97 | 94 | 93 | 94 |

1. A pharmaceutical composition comprising a compound of formula (I) or a salt thereof, and a pharmaceutically acceptable excipient as a carrier, wherein the salt is selected from the group consisting of hydrochloride and isethionate:

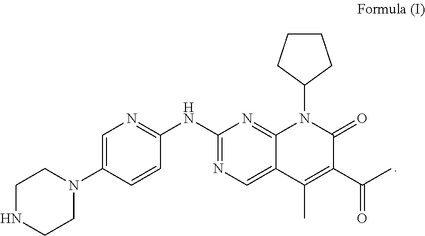 [SMILES: CC(=O)c1c(C)c2cnc(Nc3ccc(N4CCNCC4)cn3)nc2n(C2CCCC2)c1=O]

2. The pharmaceutical composition according to claim 1, wherein the compound of formula (I) or the salt thereof is present in an amount of 10%-80% by weight, relative to the total weight of the pharmaceutical composition.

3. The pharmaceutical composition according to claim 1, wherein the excipient is at least one selected from the group consisting of a disintegrant, diluent, binder, surfactant, and lubricant, and the weight percentage of each component, when present, in the pharmaceutical composition is as follows:

| | |
|---|---|
| compound of formula (I) or the | 10-80% |
| salt thereof | |
| disintegrant | 1-25% |
| diluent | 10-80% |
| lubricant | 0.1-5.0% |
| surfactant | 0-5.0% |
| binder | 0-20%. |

4. The pharmaceutical composition according to claim 3, wherein the weight percentage of each component in the pharmaceutical composition is as follows:

| | |
|---|---|
| compound of formula (I) or the | 15-60% |
| salt thereof | |
| disintegrant | 3-15% |
| diluent | 30-80% |
| lubricant | 0.1-3.5% |
| binder | 0-10% |
| surfactant | 0-5.0%. |

5. The pharmaceutical composition according to claim 4, wherein the weight percentage of each component in the pharmaceutical composition is as follows:

| | |
|---|---|
| compound of formula (I) or salt | 20-40% |
| thereof | |
| disintegrant | 3-15% |
| diluent | 55-70% |
| lubricant | 0.1-3.5% |
| binder | 0-8% |
| surfactant | 0-5.0%. |

6. The pharmaceutical composition according to claim 3, wherein the diluent is at least one selected from the group consisting of starch, powdered sugar, dextrin, lactose, pregelatinized starch, calcium hydrogen phosphate, calcium sulfate, calcium carbonate, mannitol, sorbitol and microcrystalline cellulose.

7. The pharmaceutical composition according to claim 3, wherein the binder is at least one selected from the group consisting of starch slurry, hydroxypropyl methylcellulose, hydroxypropyl cellulose, povidone, methyl cellulose, sodium carboxymethyl cellulose and polyethylene glycol.

8. The pharmaceutical composition according to claim 3, wherein the disintegrant is at least one selected from the group consisting of croscarmellose sodium, sodium carboxymethyl starch, crospovidone, dry starch and low-substituted hydroxypropyl cellulose.

9. The pharmaceutical composition according to claim 3, wherein the lubricant is at least one selected from the group consisting of magnesium stearate, stearic acid, glyceryl stearate, colloidal silica, silica, talc and glyceryl behenate.

10. The pharmaceutical composition according to claim 3, wherein the surfactant is sodium dodecyl sulfate, Tween 80 or Poloxamer 188.

11. A method of preparing a pharmaceutical composition comprising a compound of formula (I) or a salt thereof: 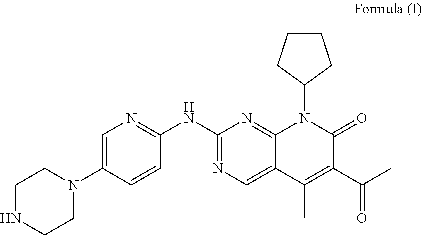 [SMILES: CC(=O)c1c(C)c2cnc(Nc3ccc(N4CCNCC4)cn3)nc2n(C2CCCC2)c1=O]
wherein the pharmaceutical composition is prepared by wet granulation, and the method comprises the following steps of:
(a) pre-mixing the compound of formula (I) or the salt thereof with a portion of an excipient in a wet mixing granulator to obtain a pre-mixture;
(b) adding a granulation liquid to granulate the pre-mixture obtained in step (a) to obtain a granule;
(c) drying the granule obtained in step (b) in a fluidized bed dryer or a drying oven to obtain a dry granule;
(d) optionally, dry screening the dry granule obtained in step (c);
(e) mixing the dry granule obtained in step (c) with the remainder of the excipient to obtain a final mixture;
(f) optionally, filling the mixture obtained in step (e) using a suitable capsule filling machine to prepare a capsule;
(g) optionally, pressing the mixture obtained in step (e) using a suitable tabletting machine to obtain a tablet core;
(h) optionally, film coating the tablet core obtained in step (g) with a film coating.

12. A method of preparing a pharmaceutical composition comprising a compound of formula (I) or a salt thereof: 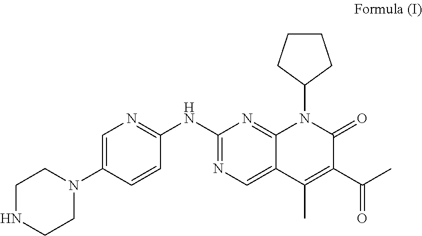 [SMILES: CC(=O)c1c(C)c2cnc(Nc3ccc(N4CCNCC4)cn3)nc2n(C2CCCC2)c1=O]
wherein the pharmaceutical composition is prepared by dry granulation, and the method comprises the following steps of:
(a) mixing the compound of formula (I) or the salt thereof with a portion of an excipient in a hopper mixer to obtain a pre-mixture;
(b) pressing the mixture obtained in step (a) in a suitable roller press machine to obtain a ribbon;
(c) crushing the ribbon obtained during step (b) into a granule by a suitable grinding or screening step;
(d) optionally, mixing the granule obtained in step (c) with the remainder of the excipient in a mixer to obtain a final mixture;
(e) optionally, filling the mixture obtained in the aforementioned step (d) using a suitable capsule filling machine to prepare a capsule;
(f) optionally, pressing the mixture obtained in the aforementioned step (d) using a suitable press tabletting to obtain a tablet core;
(g) optionally, film coating the tablet core obtained in step (f) with a film coating.

13. A method of preparing a pharmaceutical composition comprising a compound of formula (I) or a salt thereof according to claim 1, wherein the pharmaceutical composition is prepared by a method of direct mixing.

14. The preparation method according to claim 13, wherein the method comprises the following steps of:
(a) mixing the compound of formula (I) or the salt thereof with all other excipients in a hopper mixer to obtain a mixture;
(b) optionally, filling the mixture obtained in step (a) using a capsule filling machine to prepare a capsule;
(c) optionally, pressing the mixture obtained in step (a) using a suitable tabletting machine to obtain a tablet core;
(d) optionally, film coating the tablet core obtained in step (c) with a film coating.

15. The pharmaceutical composition according to claim 2, wherein the compound of formula (I) or the salt thereof is present in an amount of 20%-40% by weight, relative to a total weight of the pharmaceutical composition.

16. The pharmaceutical composition according to claim 5, wherein the diluent is at least one selected from the group consisting of lactose and microcrystalline cellulose.

17. The pharmaceutical composition according to claim 5, wherein the binder is at least one selected from the group consisting of hydroxypropyl cellulose and povidone.

18. The pharmaceutical composition according to claim 5, wherein the disintegrant is sodium carboxymethyl starch.

19. The pharmaceutical composition according to claim 5, wherein the lubricant is at least one selected from the group consisting of magnesium stearate and colloidal silica.

20. The pharmaceutical composition according to claim 5, wherein the surfactant is sodium dodecyl sulfate.
